(19) United States
(12) Patent Application Publication
(10) Pub. No.: US 2024/0055074 A1
(43) Pub. Date: Feb. 15, 2024

(54) SYSTEM AND METHOD FOR ESTIMATING MUTABILITY OF GENOMIC SEGMENTS

(71) Applicants: Mohammed KOHANDEL, Waterloo (CA); AmirHossein DAROONEH, Waterloo (CA); Michelle PRZEDBORSKI, Waterloo (CA)

(72) Inventors: Mohammed KOHANDEL, Waterloo (CA); AmirHossein DAROONEH, Waterloo (CA); Michelle PRZEDBORSKI, Waterloo (CA)

(21) Appl. No.: 18/258,286

(22) PCT Filed: Dec. 22, 2021
(86) PCT No.: PCT/CA2021/051867

§ 371 Date: Jun. 19, 2023

Related U.S. Application Data
(60) Provisional application No. 63/205,542, filed on Dec. 23, 2020.

(51) Int. Cl. G16B 20/50

(52) U.S. Cl. CPC G16B 20/50

(57) ABSTRACT

Provided are a system and method for estimating mutability of genomic segments from a reference genomic sequence. The method including: receiving the reference genomic sequence and coding regions for the genomic sequence; dividing the reference genomic sequence into genomic codons; determining an importance value for each genomic codon, the importance value representative of an estimation of the mutability of the genomic codon, the importance value including a combination of eccentricity of the genomic codon and frequency of occurrence of the genomic codon in a coding region of the genomic sequence; and outputting the importance value of each genomic codon as an estimation of the mutability of such genomic codon.

### TECHNICAL FIELD

The following relates generally to examination of genomic sequences and more specifically to a system and method for estimating mutability of genomic segments.

### BACKGROUND

In response to viral contagions, researchers attempt to develop therapeutic strategies to help mitigate the effects of the virus; including novel antiviral drugs, appropriate combinations of existing pharmaceuticals, and vaccine candidates. To ensure sustained efficacy, it is most efficacious if novel therapeutics target conserved parts of the viral genome since these segments are not considerably affected by viral mutations. Some approaches are based on sequence alignment can be used to find the conserved parts of a viral genome. These approaches generally involve performing pairwise alignment between a predecessor and a descendant sequence. While this enables the characterization of the viral mutations, these approaches generally require an extensive number of sequences that are rooted from the same ancestor and must be collected over time. Thus, when facing a novel virus or pathogen that has the potential to lead to a widespread epidemic or a global pandemic, this waiting process significantly impedes rapid development of targeted therapeutics; which could have a critical impact on case fatality rate or magnitude of an outbreak. Importantly, the mutation information can seldom be extracted directly from the mutational changes observed in other members in the virus family.

### SUMMARY

In an aspect, there is provided a method for estimating mutability of genomic codons from a reference genomic sequence, the method comprising: receiving the reference genomic sequence and coding regions for the genomic sequence; dividing the reference genomic sequence into genomic codons; determining an importance value for each genomic codon, the importance value representative of an estimation of the mutability of the genomic codon, the importance value comprising a combination of eccentricity of the genomic codon and frequency of occurrence of the genomic codon in a coding region of the genomic sequence; and outputting the importance value of each genomic codon as an estimation of the mutability of such genomic codon.

In a particular case of the method, the eccentricity of the genomic codon is determined by determining clustering of such genomic codon near boundaries of regions of the reference genomic sequence.

In another case of the method, clustering near a boundary at the end of the coding region is weighted more heavily than clustering near a boundary at the beginning of the coding region.

In yet another case of the method, the eccentricity comprises, for each coding region, a sum of a square of the distance between the position of each instance of a codon and the position of the first quarter of the coding region.

In yet another case of the method, the importance value comprises normalizing the frequency and the eccentricity.

In yet another case of the method, the importance value comprises a multiplication of a logarithmic expression of the frequency and a logarithmic expression of the eccentricity.

In yet another case of the method, the importance value is scaled between a predetermined minimum value and maximum value.

In yet another case of the method, the predetermined minimum value is 0 and the maximum value is 1.

In yet another case of the method, the method further comprising determining a mean importance value for one or more genes in the reference genomic sequence by determining an average of the constituent codons of the gene, and outputting the mean importance value.

In another aspect, there is provided a system for estimating mutability of genomic codons from a reference genomic sequence, the system comprising one or more processors in communication with a data storage and configured to execute: an input module to receive the reference genomic sequence and coding regions for the genomic sequence; a segmentation module to divide the reference genomic sequence into genomic codons; an importance module to determine an importance value for one or more genomic codons, the importance value representative of an estimation of the mutability of the genomic codon, the importance value comprising a combination of eccentricity of the genomic codon and frequency of occurrence of the genomic codon in a coding region of the genomic sequence; and an output module to output the importance value of the genomic codons as an estimation of the mutability of such genomic codon.

In a particular case of the system, the eccentricity of the genomic codon is determined by the importance module by determining clustering of such genomic codon near boundaries of regions of the reference genomic sequence.

In another case of the system, clustering near a boundary at the end of the coding region is weighted more heavily than clustering near a boundary at the beginning of the coding region.

In yet another case of the system, the eccentricity comprises, for each coding region, determining a sum of a square of the distance between the position of each instance of a codon and the position of the first quarter of the coding region.

In yet another case of the system, the importance value comprises normalizing the frequency and the eccentricity.

In yet another case of the system, the importance value comprises a multiplication of a logarithmic expression of the frequency and a logarithmic expression of the eccentricity.

In yet another case of the system, the importance value is scaled between a predetermined minimum value and maximum value.

In yet another case of the system, the predetermined minimum value is 0 and the maximum value is 1.

In yet another case of the system, the importance module further determines a mean importance value for one or more genes in the reference genomic sequence by determining an average of the constituent codons of the gene, and wherein the output module further outputs the mean importance value.

These and other aspects are contemplated and described herein. It will be appreciated that the foregoing summary sets out representative aspects of the system and method to assist skilled readers in understanding the following detailed description.

### DESCRIPTION OF THE DRAWINGS

The features of the invention will become more apparent in the following detailed description in which reference is made to the appended drawings wherein:

FIG. 1 is a block diagram of a system for estimating mutability of genomic segments, in accordance with an embodiment;

FIG. 2 is a flow chart of a method for estimating mutability of genomic segments, in accordance with an embodiment;

FIG. 3A shows a chart of a probability of appearance of each nucleotide (A—Adenine, C—Cytosine, G—Guanine, T—Thymine) in a reference SARS-CoV-2 genome sequence and a fraction of mutated nucleotides for a National Center for Biotechnology Information (NCBI) data set;

FIG. 3B shows a chart of a probability of appearance of each nucleotide (A—Adenine, C—Cytosine, G—Guanine, T—Thymine) in a reference SARS-CoV-2 genome sequence and a fraction of mutated nucleotides for a Global Initiative on Sharing Avian Influenza Data (GISAID) data set;

FIG. 4A shows diagrams of percentage of different mutations observed for each nucleotide (A, C, G, T) for the NCBI data set, where axis labels reference nucleotide substitutions, “Del” refers to deletion events, and “Ins” are nucleotide insertions;

FIG. 4B shows diagrams of percentage of different mutations observed for each nucleotide (A, C, G, T) for the GISAID data set, where axis labels reference nucleotide substitutions, “Del” refers to deletion events, and “Ins” are nucleotide insertions;

FIG. 5A shows diagrams of percentage of mutations that occur among different possible three-nucleotide sequences for the NCBI data set, where for each sequence, the mutation occurs in the central nucleotide, as indicated at the center of each plot;

FIG. 5B shows diagrams of percentage of mutations that occur among different possible three-nucleotide sequences for the GISAID data set, where for each sequence, the mutation occurs in the central nucleotide, as indicated at the center of each plot;

FIG. 6 is a chart showing a number of codon mutations associated with each nucleotide position in the SARS-CoV-2 whole genome, according to the GISAID data set, where rectangles at the bottom of the figure depict different gene regions;

FIG. 7 is a chart showing probability of nucleotide-substitution codon changes in the SARS-CoV-2 genome, based on the GISAID data set, where the y-axis corresponds to the origin codons in the reference genome and the x-axis is the destination codon;

FIG. 8 is a chart showing total number of mutations and the number of silent mutations for distinct codon types, based on GISAID data set;

FIG. 9 is a chart showing positions of two codons, CGG and TAA, in the SARS-CoV-2 reference genome, where the vertical lines are the position of the codons and the rectangles are gene regions in the sequence;

FIG. 10 is a chart showing relative density of codons in the SARS-CoV-2 genome, arranged in order of increasing codon importance, for a low mutation repetition group in the GISAID data set;

FIG. 11 are charts showing average relative density from mutation data in the GISAID data set of SARS-CoV-2 viral genes and showing average importance of SARS-CoV-2 viral genes based on the SARS-CoV-2 reference genome;

FIG. 12 is a chart showing number of mutations versus rank for all codon positions in the genome, for each codon type, obtained from the GISAID data set; and

FIG. 13 is a chart showing mutation index of each codon, with codons arranged from least to most important.

### DETAILED DESCRIPTION

Embodiments will now be described with reference to the figures. For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practised without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

Due to the urgency in controlling a highly infectious pathogen, it is imperative to have a method for extracting mutational information as quickly as possible so that therapeutic targets can be promptly identified. The present embodiments provide an approach that can advantageously extract mutational propensity of different segments of a whole genome directly from an ancestral sequence; and is thus ideal for, for example, rapid development of targeted therapeutics when a novel pathogen is identified. Using the approaches of the present embodiments, an arbitrary genomic segment can be assigned an importance value based on its repetition and spatial distribution within an ancestral genome. The importance values can then be used to estimate the mutability of segments in the whole genome, where the conserved or low mutation parts are those with a high importance value. To demonstrate the validity of this approach, example experiments applied it to the SARS-CoV-2 reference genome and show that the segments that are identified as important strongly correlate with the conserved sequences that are identified through standard mutational analysis of nearly 80,000 complete genome sequences for the virus. Particularly, the approaches of the present embodiments were used to identify conserved segments of six and seven codons in the SARS-CoV-2 genome that are potential candidates for stable siRNA-based targeted drugs.

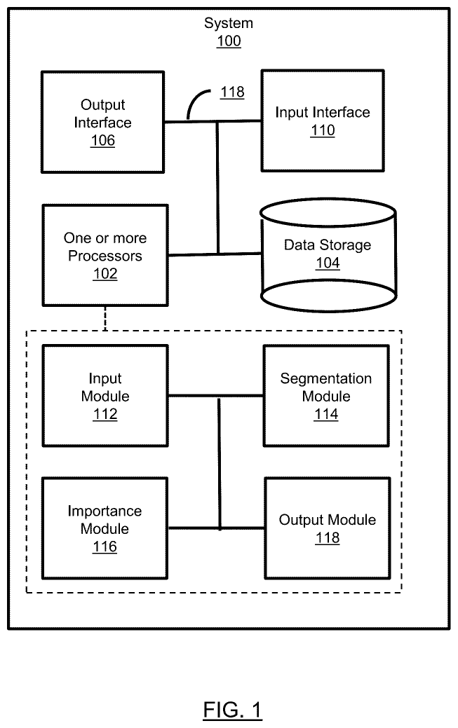

Referring now to FIG. 1, a system 100 for estimating mutability of genomic segments, in accordance with an embodiment, is shown. As understood by a person skilled in the art, in some cases, some components of the system 100 can be executed on separate hardware implementations. In other cases, some components of the system 100 can be implemented on one or more general purpose processors that may be locally or remotely distributed.

FIG. 1 shows various physical and logical components of an embodiment of the system 100. As shown, the system 100 has a number of physical and logical components, including one or more processors 102, data storage 104, an output interface 106, an input interface 110, and a local bus 118 enabling the components to communicate each other. The system 100 also includes a number of conceptual modules executed on the one or more processors 102; for example, a input module 112, a segmentation module 114, a importance module 114, and an output module 118. In an embodiment, the modules can be executed on the one or more processors 102. In other embodiments, the modules can be implemented in hardware, via a dedicated processor, or on a remote processor via a network. In some cases, the functions of the modules can be combined or further separated, as appropriate. In some cases, the output interface 106 can be in communication with a display device or other computing devices for outputting the output of the system 100. In other embodiments, non-essential elements of system 100 can be omitted or combined with other elements.

The arrangement of nucleotides in a genome sequence is not purely random. This is because physical and chemical interactions between nucleotides determine which nucleotide is more likely to be a neighbor of another nucleotide. These interactions thus give rise to order in the sequence; however, randomness is introduced by thermal fluctuations, environmental interactions, and nonequilibrium conditions arising during replication processes. The competition between order and disorder leads to some segments of the sequence being more stable, or important, than others. The present inventors recognized that this observation is very similar to patterns in written texts, in which some words are more important and responsible for conveying the meaning of a passage of text, while others are common words. Advantageously, the present inventors used techniques similar to text-mining to identify important parts in a specific genome sequence.

A text can be considered as a one-dimensional array. The appearances of a specific word are occupied by distinct positions in this discrete space. In random text, words are distributed uniformly because there is no preference for placing a word in proximity to another word and the position of a word is independent of the position of other words. Therefore, in every part of text there is a non-zero probability for finding a certain word. In contrast, in natural text, the positions of words are determined based on the grammatical rules and the context of the text, thus the position of each word strongly depends on the position of other words. For genomic sequences, the overall functionality of the sequence is synonymous with the meaning of a passage of text, and the chemical and physical interactions between nucleotides or codons are analogous to grammatical rules. The existence of short-range and long-range order causes the distribution pattern of a word to deviate from uniformity and to be clustered. The important words are more clustered than common words. By randomly shuffling the words in a passage of text, the meaning is lost and grammatical rules are also violated. Importantly, shuffling does not considerably alter the pattern of words that are distributed near uniformly, whereas the distribution of clustered words experiences a drastic change. This observation allowed the present inventors to consider clustering as a measure of importance in addition to word frequency.

Several approaches are available for ranking distinct words of a text according to their importance by identifying clustering of a word in a text. The system 100 uses an approach for characterizing genome segments based on the frequency of occurrence of the segment in a whole genome, as well as its closeness to boundaries and clustering within the genome, to associate an importance value to different genomic “words”.

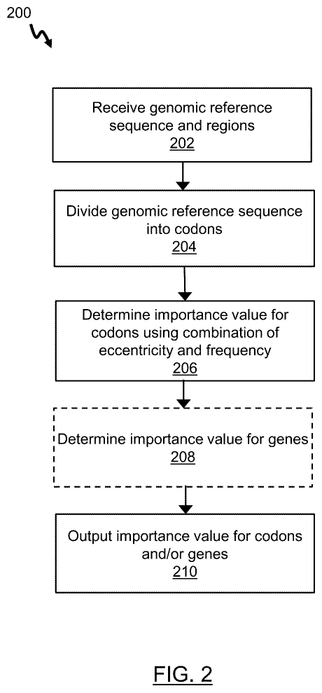

FIG. 2 illustrates a flowchart for a method for estimating mutability of genomic segments, in accordance with an embodiment. At block 202, the input module 112 receives a genomic reference sequence and coding regions for the genomic sequence.

At block 204, the segmentation module 114 divides the reference genomic sequence into genomic codons and stores the associated position of each codon in the data storage 104. In some cases, what is considered a “word” in the genomic sequence can be considered a codon; which are three-nucleotide sequences that encode for amino acids. However, in other cases, any suitable segmentation, and thus what is considered a ‘codon’, can be used. The position of the codon is taken to be the location of its first nucleotide in the genomic sequence.

At block 206, the importance module 114 determines an importance value for one or more of the codons. The importance value representative of the mutability of a particular codon in the genomic reference sequence. Like keywords in text, significant codons form clusters. Furthermore, clustering near region boundaries is taken to be more important than other places in the genomic sequence. To quantify these properties, the importance module 114 uses an eccentricity e(w) value for a word (codon) w, which is defined as follows:

$$e ( w ) = \frac{1}{R} {\sum }_{r = 1}^{R} {\sum }_{i = 1}^{{f}_{r} ( w )} {( {x}_{i} ( w ) - {m}_{r} )}^{2} \qquad ( 1 )$$

where R is the number of coding regions in the sequence and fᵣ(w) is the frequency of occurrence of w in the r-th coding region. Additionally, xᵢ(w) is the i-th position of the word in the coding region and mᵣ is the position of the first quarter of the r-th coding region. The length of the coding region varies for different genes and the information for making several proteins may be coded in a gene. Identifying the coding regions can use any suitable approach, such as through experimentation performed on the sequence. The convention of Equation (1) implies that clustering at the end of a coding region is more important than at the beginning because, as described herein, the present inventors determined that there is a higher correlation with relative density and mutation index at the end of a region. When a coding region is formed by joining several disconnected smaller regions, the eccentricity from the first quarter of all smaller regions can be taken into account. Advantageously, this approach can include all regions that code a protein without regarding its length; considering any portion in a text that has a meaning. The definition of eccentricity in Equation (1) may not be able to capture clustering near mᵣ; however, in the present embodiments, if a codon is clustered, its distance to boundaries has less importance.

In addition to the eccentricity, the importance module 114 determines the importance value of a codon using a determination of frequency of occurrence of the codon in a coding region of the genomic sequence. It is generally accepted that bias in repetition of codons has biological consequences. Thus, the frequency of occurrence is combined with eccentricity by the importance module 114 to generate a unified rule for assigning importance to codons. In a particular case, determining of the importance i(w) of a codon w can include:

$$i ( w ) = ln ( 1 + \sqrt{\frac{f ( w )}{{Σ}_{v} f ( v )}} ) \times ln ( 1 + \frac{e ( w )}{{Σ}_{v} e ( v )} ) \qquad ( 2 )$$

where f(w) is the frequency of occurrence of codon w and e(w) is its eccentricity, calculated using Equation (1). This form is advantageous for determining the importance because, by normalizing both the frequency and the eccentricity, their values become comparable to each other. However, any suitable combination of eccentricity and frequency can be used to determine the importance value. In the case of Equation (2), the square root appears in the first logarithm to account for the fact that the frequency values are distributed over a larger scale than the eccentricity values. Based on the empirical data, taking the square root of normalized frequency reduces the variation in the frequency of different codons to one order of magnitude, like their eccentricity variation. Thus, incorporation of the square root inhibits the codon frequency from dominating the importance. The logarithm is further used to reduce large differences. Moreover, by multiplication of the two logarithmic expressions, all combinations between frequency and eccentricity are taken to account.

In some cases, the importance module 114 can scale the importance measure by an arbitrary or selected factor. Thus, in some cases, to draw conclusions about which codons are most important within a given genome, the importance module 114 can examine how a given codon compares with all other codons in that genome. In example experiments described herein, eccentricity of codons are determined over 28 coding regions in the SARS-CoV-2 genomic sequence. Some coding regions have overlap, therefore, in some cases, a codon-position may contribute to the determination of importance of more than one codon through the eccentricity.

At block 208, in some cases, the importance module 114 can determine a mean importance value for genes in the genomic reference sequence by determining an average of the importance values of the constituent codons associated with each gene.

At block 210, the output module 118 outputs the importance value for each codon, the mean importance value for one or more genes, or both, to the output interface 106 and/or the data storage 104.

The present inventors conducted example experiments to illustrate the substantial advantages of the present embodiments. In example experiments, nearly 80,000 complete genome sequences for SARS-CoV-2 were curated from two public repositories and then subsequently analyzed. A reference sequence, NC_045512.2, was also obtained. This is the first genomic sequence for SARS-CoV-2, whose origin is Wuhan, China, and it was made publicly available in January 2020. The reference sequence is comprised of 29,903 nucleotides. The sequence contains 28 coding regions that specifically code the virus proteins. These coding regions are encompassed in 10 genes: ORF1ab, S, ORF3a, E, M, ORF6, ORF7ab, ORF8, N, ORF10. These 10 genes occupy approximately 97.86% of the virus genome. The longest gene is ORF1ab, with a length of 21,290 nucleotides, while the shortest gene, ORF10, has length 117 nucleotides.

The genomic sequences from both data sets were aligned with the reference sequence, NC_045512.2. To avoid overlap between the two data sets, and to facilitate comparison of the results, the genomic analysis by the system 100 was performed independently for each data set. Based on alignments, any certain change in the genome sequences with respect to the reference sequence, including nucleotide insertions, deletions, and substitutions, were extracted and stored into a master file. This master file contained the mutation information for each sequence, as well as the specific nucleotide change and its position in the reference genome.

During empirical analysis, the number and type of mutations in the SARS-CoV-2 genome were investigated. Changes were investigated at the individual nucleotide level, including the distribution of changes at different positions in the genome. Changes at the codon level were also investigated, including insertion, substitution, and deletion mutations, and whether these mutations cause changes at the protein level.

57,939 mutations were investigated in a curated National Center for Biotechnology Information (NCBI) data set and 674,800 in a curated Global Initiative on Sharing Avian Influenza Data (GISAID) data set. In the NCBI data set, 48,554 or 83.8% of the mutations occurred in coding regions, while 561,195 or 83.2% were in coding regions for the GISAID data set. The mutation rate per nucleotide per generation, calculated as the number of mutations per number of sequences and per sequence length, is approximately 2.75×10⁻⁵ and 3.02×10⁻⁵, respectively, for the NCBI and GISAID data sets. Thus, the probability of mutation in the SARS-CoV-2 genome is low in comparison with other RNA viruses, which accounts for the stability of the SARS-CoV-2 virus and the emergence of the associated pandemic.

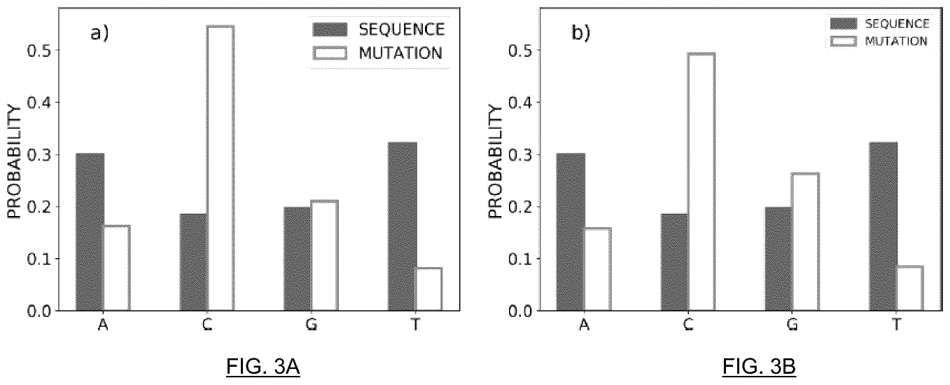

The frequency of nucleotide occurrence in a genome is a basic specification for any genomic sequence, and it is related to the effective energy usage by organisms in their duplication process. In FIGS. 3A and 3B, the probability of occurrence of each nucleotide in the reference sequence NC_045512.2 is shown in the NCBI data set and the GISAID data set, respectively; calculated as the fraction of the whole genome comprised of each nucleotide. Each nucleotide type does not appear with the same frequency of occurrence. In particular, Thymine (T) occurs most frequently 32.08% in the reference sequence, while Cytosine (C) appears least often, 18.36%. In addition, in FIGS. 3A and 3B, the fraction of mutated nucleotides is plotted, which evidently varies significantly among the different bases. These results indicate that mutations do not occur randomly, since in the latter case, each nucleotide type would have the same probability of mutation and occurrence. Interestingly, Cytosine has a significantly higher probability of mutation, despite being the lowest occurring nucleotide (54.51% in the NCBI and 49.23% in the GISAID sequences). In contrast, Thymine, which occurs most frequently, has the lowest probability of mutation, 8.17% and 8.41% for the NCBI and GISAID datasets, respectively.

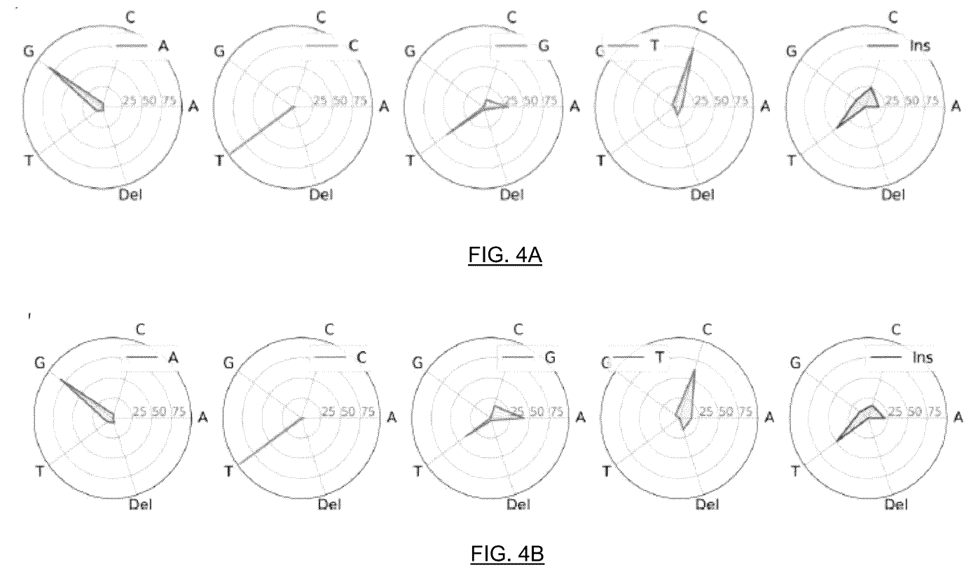

The percentage of different mutations in the SARS-CoV-2 genome that are observed for each nucleotide type is shown in FIGS. 4A and 4B for the NCBI data set and the GISAID data set, respectively. Overall the pattern of evolution, i.e. GC→AT, is apparent in the virus mutations. Interestingly, it is seen that the results for both the NCBI and GISAID data sets are quite similar, with slight differences in the distributions for Cytosine, Thymine, and nucleotide insertions. From this figure, it is seen that for each nucleotide, there is a distinct destination for substitution mutations. Specifically, the most probable mutations are: A→G (83.16% and 81.18%), C→T (96.72% and 95.95%), G→T (57.01% and 38.77%) and T→C (76.47% and 62.70%). In contrast, it is seen that for the insertion mutations, all nucleotide types have a considerable probability, while Thymine has the highest chance (43.75% and 49.76%). The first and second values in the parentheses are extracted from the NCBI and GISAID datasets respectively.

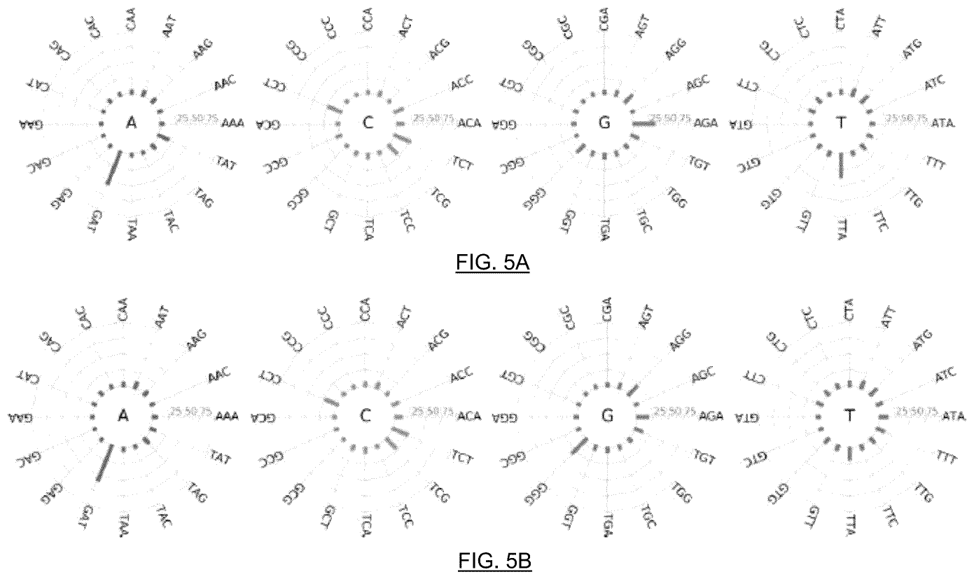

All mutated nucleotides have a neighbor on both their left and right sides. To determine whether all the corresponding nucleotide sequences have equal propensity for mutation, the percentage of mutations that are observed for each of the 16 possible nucleotide sequences, for each mutated nucleotide, is plotted in FIGS. 5A and 5B for the NCBI data set and the GISAID data set, respectively. As shown, for all four mutated central nucleotides, the distribution is not uniform among the different nucleotide sequences. This indicates that certain nucleotide sequences have a significantly higher propensity for mutation than others. For example, Cytosine is more likely to mutate when it is preceded by another Cytosine or a Thymine, with the highest probability occurring when it is also succeeded by a Thymine. Specifically, for TCT, the mutation percentage is 24.22% and 22.51% for the NCBI and GISAID, respectively. Interestingly, overall, a nucleotide has a considerable probability of mutation if it has Guanine, Adenine, or Thymine, but not Cytosine, in its immediate vicinity.

The nucleotide sequences in FIGS. 5A and 5B each occur at several positions within the SARS-CoV-2 whole genome sequence. Though almost all such positions for each nucleotide sequence are observed to be mutated, the number of mutations is not uniform across all positions along the sequence. In TABLE 1, the top ten positions with the highest frequency of substitution mutation observed in the GISAID data set are reported. TABLE 1 illustrates that some positions along the whole genome are indeed significantly more predisposed for mutation, with the top five positions comprising over 37% of all the mutations in the genome.

TABLE 1

| | | | Left | Right |
|---|---|---|---|---|
| Position | Nucleotide | Repetition | Neighbour | Neighbour |
| 23403 | A | 57221 | G | T |
| 14408 | C | 56993 | C | T |
| 3037 | C | 56957 | T | T |
| 241 | C | 55901 | T | G |
| 28881 | G | 24173 | A | G |
| 28882 | G | 24125 | G | G |
| 28883 | G | 24117 | G | G |
| 25563 | G | 16264 | A | A |
| 1059 | C | 12614 | A | C |
| 11083 | G | 8286 | T | T |

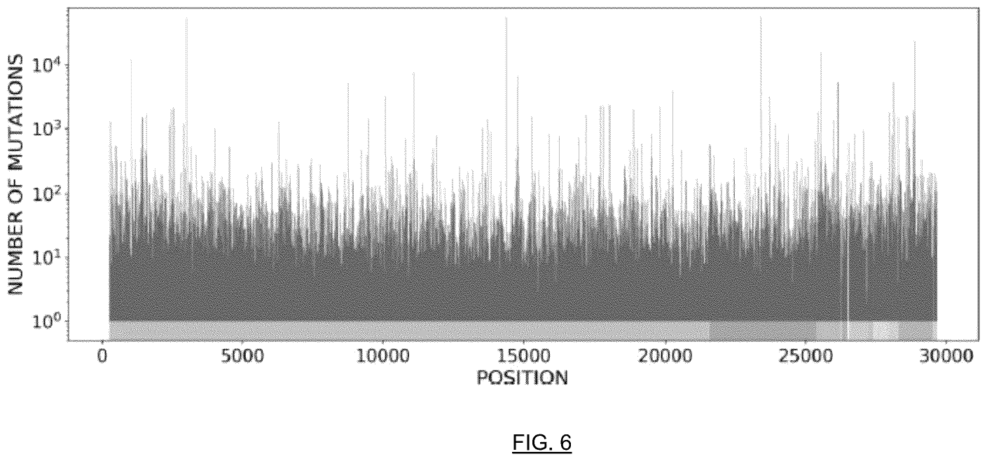

Most of the SARS-CoV-2 genome is occupied by codons, which are sequences of three nucleotides that code for specific amino acids during protein synthesis. Importantly, the codons and the corresponding protein structure of the SARS-CoV-2 virus may change due to mutations in the nucleotide sequences. To analyze the impact of the observed nucleotide mutations on the synthesis of viral proteins, the underlying distribution of codons in the SARS-CoV-2 reference sequence, NC_045512.2, was examined. It was determined that there is a non-uniform distribution in the frequency of occurrence of each codon. The number of codon mutations in each nucleotide position of the whole genome was determined and presented in FIG. 6 for the GISAID data set. The distribution of mutations is non-uniform over the genome, displaying some background periodicities. Interestingly, several of the codon positions, which include nucleotides with more than 50,000 mutations, are identified. These nucleotides are in accordance with the results in TABLE 1.

Building on these results, the example experiments investigated the distribution in the number of codon mutations along the SARS-CoV-2 genome in the GISAID data set. It was found that the distribution function behaves non-monotonically as a function of the number of mutations, and it peaks between approximately 8 and 32 codon mutations. The number of positions in the SARS-CoV-2 genome with a given number of codon mutations increases linearly when the number of mutations is small (≤2³); however, when the number of mutations exceeds ≈2³, the number of positions with a given number of codon mutations is inversely related to the mutation repetition number. This power-law behaviour emphasizes that the evolution of the genome is not a purely random process, but rather, it obeys some universal physical rules.

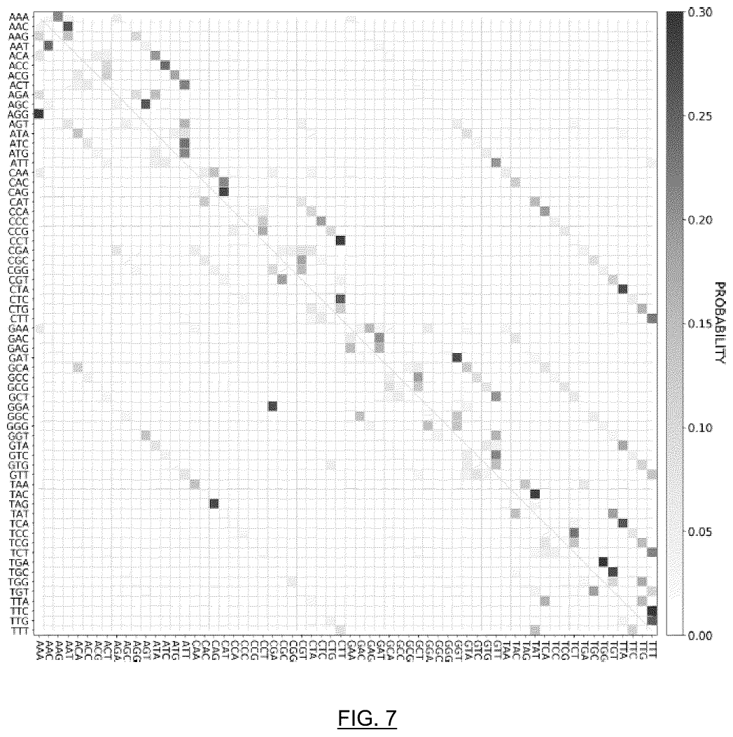

While the evolution of the SARS-CoV-2 genome is not a purely random process, examining the total number of codon mutations does not give insight into the nature of the mutations that occur. To gain further insight into the specific codon mutations, examination was conducted of the probability for different possible nucleotide-substitution codon changes, calculated based on the frequency of the observed mutation in the GISAID data set. The results are depicted in FIG. 7. The probability of each codon change is nonzero for only a small fraction of the possible codon changes. Interestingly, the codon changes with higher probability of mutation follows, on average, a series of single straight lines that originate at the top left and end at the bottom right of the plot. Each line corresponds to a specific position (first, second, or third) in the codon that is mutated, and the mutation is repetitive. For example, for the lowest line emerging from the left of FIG. 7, at the codon GAA, the first nucleotide is mutated to an Adenine for the codons beginning with Guanine, and to a Cytosine for the codons beginning with a Thymine. The ten most frequently observed nucleotide-substitution codon changes exhibited in FIG. 7 are listed in TABLE 2. Comparison of TABLE 1 and TABLE 2 reveals that some of the nucleotide positions in the SARS-CoV-2 genome with the highest probability for mutation are not involved in codon changes, for example position 241.

TABLE 2

| Position | Origin | Destination | Repetition |
|---|---|---|---|
| 23402 | GAT | GGT | 57221 |
| 14407 | CCT | CTT | 56966 |
| 3035 | TTC | TTT | 56956 |
| 28880 | AGG | AAA | 24088 |
| 28883 | GGA | CGA | 24085 |
| 25561 | CAG | CAT | 16223 |
| 1058 | ACC | ATC | 12605 |
| 11081 | TTG | TTT | 8173 |
| 14803 | TAC | TAT | 6524 |
| 28143 | TTA | TCA | 5385 |

As exhibited in FIGS. 4A and 4B, deletion mutations also occur in the SARS-CoV-2 genome. Deletion mutations, which result in the removal of segments of the genome, can cause some adjacent codons to merge into each other, potentially forming a new codon. In TABLE 3, provided are the ten most frequent deletion mutations that were observed in the SARS-CoV-2 genome in the GISAID data set. Importantly, it is seen that several of the most frequent deletion events lead to a new codon, which can change the encoded amino acid, and potentially lead to protein-level changes in the virus. It was also found that there are very rare cases in which the number of adjacent nucleotide deletions is not a multiplicative factor of three. While rare, such deletions can dramatically change the protein sequence. Insertion mutations were found to be rare, accounting for less than 0.28% of all nucleotide mutations. However, while rare, such mutations can profoundly affect the sequence of viral proteins encoded by the SARS-CoV-2 genome.

TABLE 3

| Position | Origin | Destination | Repetition |
|---|---|---|---|
| 1604 | AATGAC | A--AC | 1552 |
| 686 | AAGTCATTT | ----- | 237 |
| 21989 | GTTTAT | GT--T | 69 |
| 515 | GTTATG | ----- | 56 |
| 506 | CATGGTCATGTTATGGTT | CA--------T | 54 |
| 6329 | TCAAATTCG | ----- | 44 |
| 509 | GGTCATGTTATG | G-------TG | 43 |
| 28089 | GGTTCTAAA | G------AA | 26 |
| 21980 | TTTTTGGGTGTTTAT | TT-------T | 24 |
| 671 | TACGGCGCCGATCTA | T------TA | 23 |

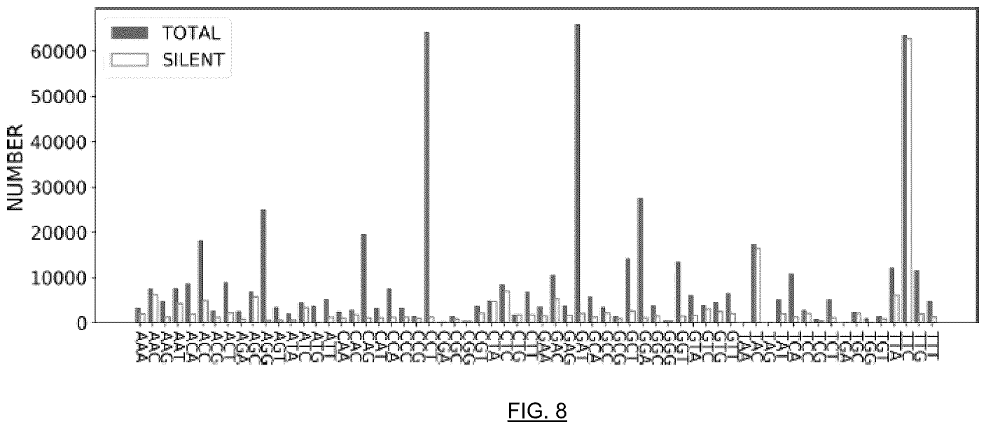

Substitution mutations may be silent, which corresponds to the case where the codon changes but it does not alter the encoded amino acid. This is possible since most amino acids can be encoded by more than one distinct codon. Thus, if codon mutations result in a degenerate codon for a specific amino acid, changes will not be observed at the level of amino acids, and consequently, at the protein level. In FIG. 8, there is a comparison of the number of silent mutations to the total number of mutations for each codon in the SARS-CoV-2 genome, obtained from the GISAID data set. On average, silent mutations make up a small number of total mutations for most of the codons. However, some codons have a tendency to undergo mostly silent mutations. For example, the codon TTC was observed to undergo a total of 63,555 mutations, and more than 98.7% of them are silent.

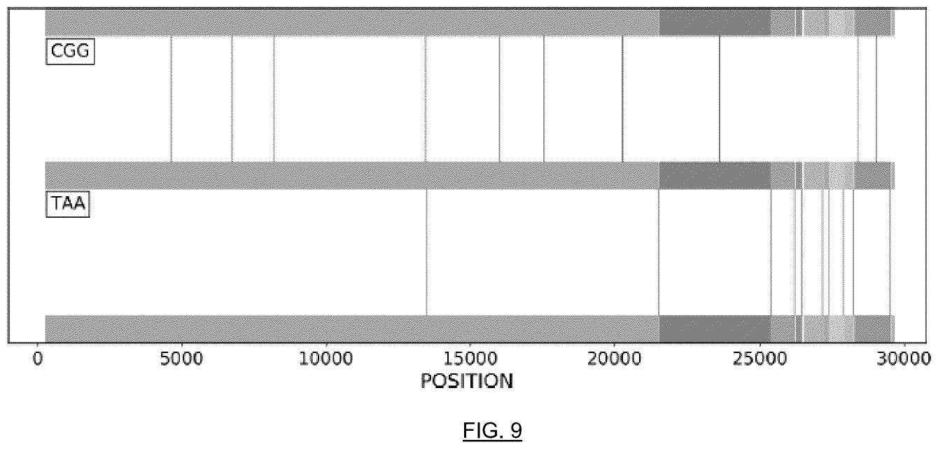

Using the method 200, the example experiments sought to determine the importance of different codons in the SARS-CoV-2 genome and use this to quantify the importance of each gene. To this end, we began by investigating the positions of each codon in the reference genome, which are depicted in FIG. 9 for the codons CGG and TAA. While these two codons have approximately the same frequency of occurrence (11 and 10 times, respectively), their distribution along the genome is markedly distinct, with the codon CGG uniformly appearing in only three distinct genes. In contrast, the codon TAA appears toward the end of most coding regions in the genome and forms a cluster toward the end. These differences in the distribution of positions complies with the importance of the stop codon, which plays a key role in protein synthesis by terminating the decoding process.

To further quantify how the distribution of codon positions is related to the importance of the codon in the genome, the eccentricities for all codons in the SARS-CoV-2 reference genome were determined, which are depicted in FIG. 9. The three codons GGG, CCC and GTC have the highest value of normalized eccentricity, 1.87×10⁻², 1.82×10⁻² and 1.77×10⁻², respectively. In FIG. 9, the codons are ordered based on their frequency of occurrence in the genome, which is also shown in the plot for comparison. The importance of each codon in the plot, calculated using Equation (2) for the reference genome, which depends on both the eccentricity and the frequency of occurrence in the genome.

As shown in FIG. 9, almost all occurrences of codons in different positions of the genomic sequence experience at least one mutation in the GISAID data set. Given the non-uniform distribution of mutation numbers across nucleotide positions, it is useful to classify the codons into low repetition and high repetition mutation groups. This is performed by segregating the codon-position pairs with a mutation repeat less than a threshold value into the low repetition group and the remaining pairs into the high repetition group. In this example, the threshold value is eight, which leads to two groups with nearly equal number of codons. To quantify the degree of mutations in all codon-position pairs along a genome, a relative density of a codon w, denoted by r(w), is defined as follows:

$$r ( w ) = \frac{{f}_{low} ( w )}{f ( w )} \qquad ( 3 )$$

where f_{low}(w) is the number of positions in which codon w has a low number of mutations (in an example, defined to be less than eight), and f(w) is the frequency of occurrence of codon w in the genome. Since a codon-position pair is either mutated a low number of times or a high number of times, the relative density varies between zero and one for each codon w, where a value of one indicates that all codon-position pairs for codon w experience a low number of mutations.

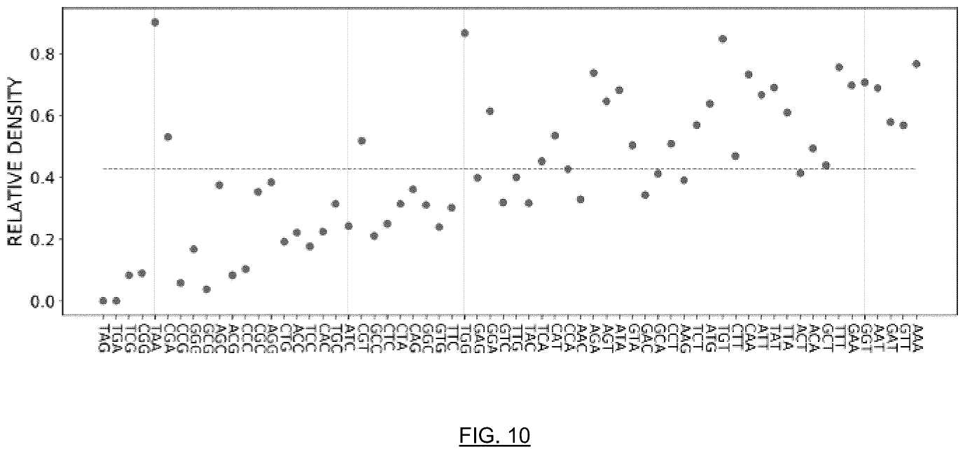

The relative density of codons was plotted in FIG. 10, obtained by applying Equation (3) to the GISAID data set. In the figure, the codons are arranged from least to most important, where the codon importance was calculated by applying Equation (2) to the SARS-CoV-2 reference genome. From the figure, it is clear that there is a strong positive correlation between codon importance and relative density. To quantify the trend, a Pearson correlation was determined between the codon importance and its relative density in the low repetition group, and found it to be greater than 0.68. Advantageously, these results show that mutation information can be inferred directly from the codon importance values associated with the reference genome.

To validate the results, the example experiments compared the results with results obtained from a random sequence with the same distribution of nucleotides and genomic structure. In a random sequence, the frequency of appearance of a three nucleotide segment is proportional to the product of the probability of its constituents, i.e. fᵣₐₙ (w=x₁x₂x₃)˜p(x₁)p(x₂)p(x₃). One can eliminate the effect of bias in usage of nucleotides by replacing the frequency in Equation (2) with the relative frequency of a codon, f(w)/fᵣₐₙ(w). This reduces the Pearson correlation between importance and relative density in the reference sequence to nearly 0.5. Once repeated on the same calculation with a random sequence, the Pearson correlation becomes very close to zero. This implies that the codon arrangement in the reference sequence is not random and instead obeys a kind of order.

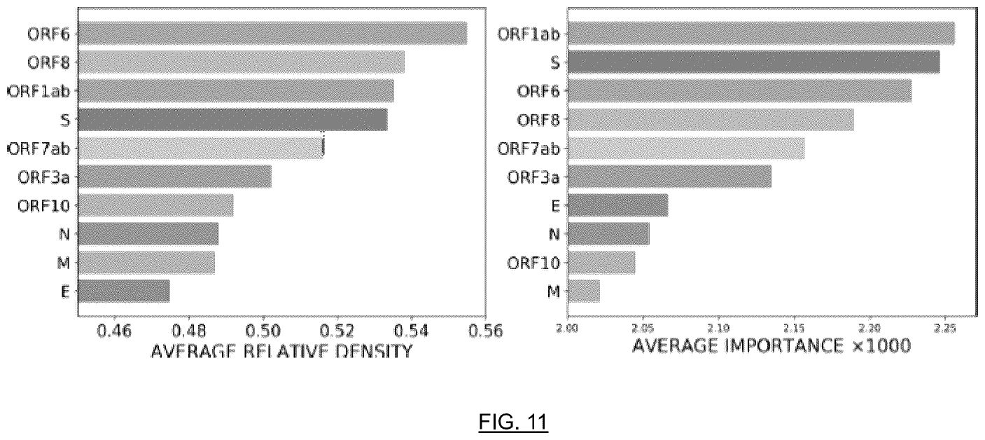

Building on these results, the SARS-CoV-2 genes were ranked according to the average of the relative density and importance of their constituent codons. The results are plotted in FIG. 11 and show that the two ranking schemes are strongly correlated with each other. To quantify the correlation, the Pearson correlation coefficient was determined for the two ranked lists, which gave a value of greater than 0.91. These results confirm that the importance of viral genes, calculated from a reference genome using the method 200, can be used to determine which genes have a higher probability for mutation and those which are likely to be conserved. Furthermore, strong correlations are also observed between these two measures for the coding regions of the genome where the value of the Pearson correlation coefficient is 0.90.

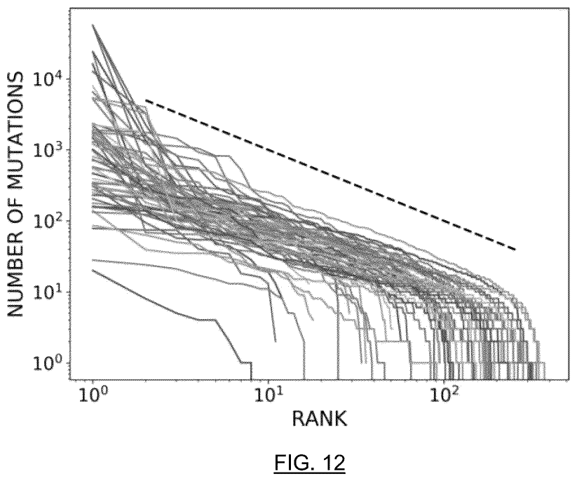

Given that each codon position is associated with a different number of mutations, embodiments of the present disclosure determine a statistical quantity, referred to as a mutation index, that characterizes the overall set of mutations for each codon type. Since the set of mutation values for each codon follows Zipf's law (frequency distribution of alleles obeys a power law relationship), the median mutation number can be used as the mutation index. In FIG. 12, the mutation index of each codon was plotted in relation to the codon importance. Increasing importance values are seen to correspond to a lower mutation index. Advantageously, this implies that there is a strong negative correlation between these two quantities. To quantify the correlation, the Pearson correlation coefficient between the codon mutation index and importance values and found it to be greater than 0.70 in magnitude.

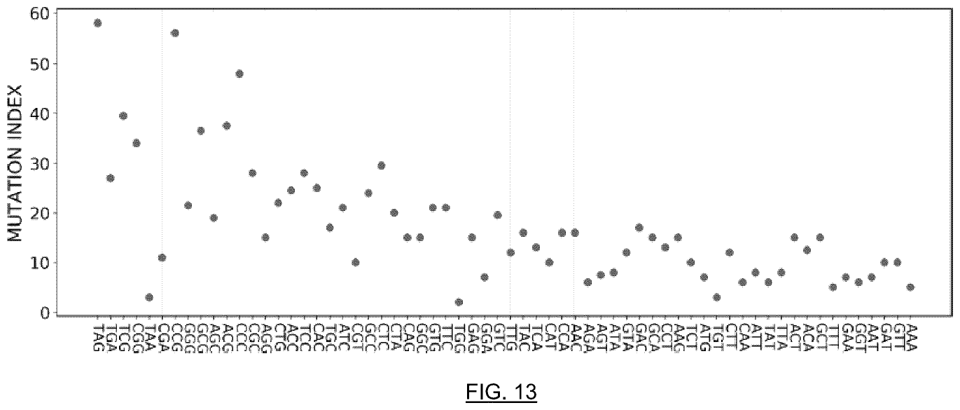

The mutation index is defined for any specific part of genome by averaging the mutation indices of the constituents of that segment. Using this approach, the mutation index, based on the mutation data from the GISAID data set, was determined for each of the SARS-CoV-2 genes in the reference genome. Similarly, the importance of each gene was determined by taking the average of the importance values of their constituent codons in the reference genome. To quantify the strong negative correlation between these two measures, the Pearson correlation coefficient was determined to have a value of −0.92. To illustrate the negative correlation, the average mutation index and the average importance of the SARS-CoV-2 viral genes are plotted in FIG. 13. In the figure, the genes are ranked in order of increasing mutation index and decreasing importance.

Notably, the average mutation index and the average importance of the coding regions of the SARS-CoV-2 viral genes are also highly correlated. To quantify the degree of correlation, the Pearson correlation coefficient was determined between these two measures for all of the coding regions in the SARS-CoV-2 genome, and found it to be −0.91; indicating a strong negative correlation with a similar magnitude as the correlation for the viral genes. Thus, the genes can be ranked either from most to least important, or by increasing mutation index, to find that the difference between the two ranking schemes is negligible for most coding regions.

These results indicate that the importance of codons determined in the method 200 is strongly negatively correlated with their propensity for mutation. Thus, by analyzing the importance of any segment of a reference genome, the system 100 can immediately infer the mutability of this segment. Specifically, in the case of novel viruses and pathogens, an index of mutability can be determined by calculating the importance of different segments from the first identified genome, without having to wait to gather genomes from other infected individuals to analyze the mutations. This is a significant result because by requiring information about only one infection, the time it takes to develop targeted therapeutics for a novel pathogen is drastically reduced. The system 100 can thus be immediately used to develop stable drugs that are based on short interfering RNA (siRNA) by targeting important genome segments of the pathogen that have low propensity for mutation. With respect to the SARS-CoV-2 in the example experiments, the top ten important segments, which consist of six and seven codons, respectively, are provided in TABLE 4 and TABLE 5. Inspection of TABLE 4 and TABLE 5 confirms that non-structural protein 3 (nsp3) and spike protein (S) are the best targets for antiviral drugs. It should be noted that in TABLE 4 and TABLE 5, the importance values of the codons are scaled between 0 and 1 using Equation (4):

$${i}_{scaled} ( w ) = \frac{i ( w ) - {i}_{min}}{{i}_{max} - {i}_{min}} \qquad ( 4 )$$

where iₘᵢₙ(iₘₐₓ) is the minimum (maximum) importance value for the codons. Following the convention above, the importance of a segment is the average of the importance of its constituent codons.

As illustrated in the example experiments, the method 200 can be advantageously used to identify the most important segments in a genome sequence. While the example experiments described genome segment length of codons, it is to be understood that the length of the segment is arbitrary, thus the method 200 can be used to identify important segments across various suitable length scales; for example, at the level of codons, genes, or gene coding regions. The example experiments illustrate that the method 200 can be used to identify important segments in the SARS-CoV-2 reference genome directly from codon frequencies of occurrence and spatial distribution along the sequence. Significantly, it showed that the segments that were identified as important strongly correlate with the conserved sequences identified through pairwise alignment of the reference genome with nearly 80,000 SARS-CoV-2 predecessor complete genome sequences.

In the empirical mutational analysis of the example experiments, over 732,000 nucleotide mutations in the SARS-CoV-2 genome data set were identified, and over 83% of these mutations were found to occur in coding regions of the virus, potentially leading to changes at the protein level. While characterizing the nucleotide mutations, the example experiments found that the mutation frequency was negatively correlated with the frequency of occurrence of each nucleotide in the reference SARS-CoV-2 genome. Furthermore, the example experiments found that for each nucleotide, there was one distinct destination for substitution mutations, which were the most common type of mutations observed in the data set. Overall, nucleotide mutations tended to increase the AT content of the genome. Looking at the neighbours of mutated nucleotides, the example experiments further determined that some nucleotide sequences have a higher propensity for mutation than others. Notably, a nucleotide with a Guanine, Adenine, or Thymine, but not Cytosine, in its immediate vicinity was found to have a considerably higher probability of mutation.

Two measures, the relative density and the mutation index, were used to characterize the mutations observed in different segments of the genome. It was determined that the relative density of a genome segment in a low mutation group was strongly correlated with its importance value; which indicates that important segments tended to have a low number of mutations. This correlation was observed to hold at the codon and gene levels, and also for the coding regions of each gene. The determination that Zipf's law holds between the number of mutations of a codon position and its rank in the set of mutations for each codon enabled the definition of the mutation index to characterize the mutability of different codons. It was found that the mutation index of different genome segments was highly negatively correlated with the importance of these segments, with the strong correlation observed at the codon and gene levels, as well as for the coding regions of each gene. This result strengthens the assessment that important segments have a lower number of mutations. Advantageously, the present embodiments can be used to identify potential candidates for, for example, stable siRNA-based targeted drugs that can inhibit the production of viral proteins. For the purposes of illustration, in the example experiments, the method 200 was used to identify the most important six and seven codon sequences from the reference SARS-CoV-2 genome, which are most likely to be stable against future genomic mutations and may therefore be candidates for siRNA-based antiviral drugs.

In the example experiments, the SARS-CoV-2 genes that were identified by method 200 confirm pathogenesis of the virus in humans. Specifically, the genes encoding the structural proteins (S, E, M, N) and the gene ORF1ab, which encodes several non-structural proteins, were identified to be important by application of the method 200 to the reference SARS-CoV-2 genome. Indeed, these proteins are thought to play a crucial role in the pathogenesis of the virus. In addition, genes that encode accessory proteins (ORF6, ORF8, ORF7ab, ORF3a, ORF10) were identified as highly important by the method 200. These proteins are thought to play a role in counteracting the host's innate immune system. The SARS-COV-2 virus is known to induce an innate immune response, including the release of pro-inflammatory cytokines such as TNF-α, IL-1, and IL-6). This inflammatory response can lead to a cytokine storm, resulting in severe COVID-19 disease conditions and a high fatality rate.

Importantly, in the example experiments, when the method 200 was used to identify genomic segments with six codons as potential targets for siRNA-based therapeutics, one of the identified segments corresponds to a nonhuman-like epitope of the SARS-CoV-2 spike glycoprotein. Thus, the output can be used for the development of an siRNA-based therapeutic that can target an epitope of a structural SARS-CoV-2 protein, without the risk of inducing an autoimmune response and severe disease outcome.

Advantageously, the method 200 enables rapid identification of segments of a whole genome that are likely to remain conserved during future genomic mutation events. Importantly, these segments are identified from a reference (or ancestral) genome. Thus, the method 200 eliminates the need to wait for the collection and analysis of predecessor whole genome sequences. This not only reduces the cost but is also crucial for a timely response to highly infectious novel pathogens that have the potential to cause widespread epidemics or global pandemics. Further advantageously, the method 200 can be applied to a wide range of pathogens; for example, for the identification of novel therapeutic strategies to help overcome antimicrobial resistance, which is considered one of the biggest threats to global health.

Although the foregoing has been described with reference to certain specific embodiments, various modifications thereto will be apparent to those skilled in the art without departing from the spirit and scope of the invention as outlined in the appended claims. The entire disclosures of all references recited above are incorporated herein by reference.

1. A method for estimating mutability of genomic codons from a reference genomic sequence, the method comprising:
receiving the reference genomic sequence and coding regions for the genomic sequence;
dividing the reference genomic sequence into genomic codons;
determining an importance value for one or more of the genomic codons, the importance value representative of an estimation of the mutability of the genomic codon, the importance value comprising a combination of eccentricity of the genomic codon and frequency of occurrence of the genomic codon in a coding region of the genomic sequence; and
outputting the importance value of each of the one or more of the genomic codons as the estimation of the mutability of such genomic codon.

2. The method of claim 1, wherein the eccentricity of the genomic codon is determined by determining clustering of such genomic codon near boundaries of regions of the reference genomic sequence.

3. The method of claim 2, wherein clustering near a boundary at the end of the coding region is weighted more heavily than clustering near a boundary at the beginning of the coding region.

4. The method of claim 3, wherein the eccentricity comprises, for each coding region, a sum of a square of the distance between the position of each instance of a codon and the position of the first quarter of the coding region.

5. The method of claim 1, wherein the importance value comprises normalizing the frequency and the eccentricity.

6. The method of claim 1, wherein the importance value comprises a multiplication of a logarithmic expression of the frequency and a logarithmic expression of the eccentricity.

7. The method of claim 1, wherein the importance value is scaled between a predetermined minimum value and maximum value.

8. The method of claim 1, wherein the predetermined minimum value is 0 and the maximum value is 1.

9. The method of claim 1, further comprising determining a mean importance value for one or more genes in the reference genomic sequence by determining an average of the constituent codons of the gene, and outputting the mean importance value.

10. A system for estimating mutability of genomic codons from a reference genomic sequence, the system comprising one or more processors in communication with a data storage and configured to execute:
an input module to receive the reference genomic sequence and coding regions for the genomic sequence;
a segmentation module to divide the reference genomic sequence into genomic codons;
an importance module to determine an importance value for one or more of the genomic codons, the importance value representative of an estimation of the mutability of the genomic codon, the importance value comprising a combination of eccentricity of the genomic codon and frequency of occurrence of the genomic codon in a coding region of the genomic sequence; and
an output module to output the importance value of each of the one or more of the genomic codons as the estimation of the mutability of such genomic codon.

11. The system of claim 10, wherein the eccentricity of the genomic codon is determined by the importance module by determining clustering of such genomic codon near boundaries of regions of the reference genomic sequence.

12. The system of claim 11, wherein clustering near a boundary at the end of the coding region is weighted more heavily than clustering near a boundary at the beginning of the coding region.

13. The system of claim 12, wherein the eccentricity comprises, for each coding region, determining a sum of a square of the distance between the position of each instance of a codon and the position of the first quarter of the coding region.

14. The system of claim 10, wherein the importance value comprises normalizing the frequency and the eccentricity.

15. The system of claim 10, wherein the importance value comprises a multiplication of a logarithmic expression of the frequency and a logarithmic expression of the eccentricity.

16. The system of claim 10, wherein the importance value is scaled between a predetermined minimum value and maximum value.

17. The system of claim 16, wherein the predetermined minimum value is 0 and the maximum value is 1.

18. The system of claim 10, wherein the importance module further determines a mean importance value for one or more genes in the reference genomic sequence by determining an average of the constituent codons of the gene, and wherein the output module further outputs the mean importance value.
